(19) United States
(12) Patent Application Publication
(10) Pub. No.: US 2015/0072026 A1
(43) Pub. Date: Mar. 12, 2015

(54) Anti-inflammatory ion and uses thereof

(71) Applicant: Yunyuan Li, Vancouver (CA)

(72) Inventor: Yunyuan Li, Vancouver (CA)

(21) Appl. No.: 14/020,787

(22) Filed: Sep. 7, 2013

(51) Int. Cl. A61K 33/02; C01C 1/16

(52) U.S. Cl. CPC A61K 33/02; C01C 1/164
USPC 424/720; 424/719; 423/470

(57) ABSTRACT

The present invention related to a pharmaceutically acceptable amount of ammonium salt as an anti-inflammation agent thereof, and a pharmaceutical composition, a functional cosmetic composition, a health food, health beverages, a food additive and animal feeds containing the same.

### CROSS-REFERENCE TO RELATED APPLICATIONS

This application claims the benefit of U.S. Provisional Patent Application No. 61/698732, filed Sep. 10, 2012, entitled “anti-inflammatory ion and uses thereof”

### TECHNICAL FIELD OF INVENTION

The present invention relates to an anti-inflammatory ammonium ion (NH₄⁺) comprised in any compound (ammonium salt). This specification also describes the use of compound consist of NH4⁺ for treating different inflammatory diseases with pharmaceutically effective amounts of a formulation comprising NH₄⁺ compound.

### BACKGROUNDS OF THE INVENTION

Inflammation is an innate and adaptive immune response triggered by stimuli such as infection, tissue injury and autoantibody. Inflammation can be classified into acute and chronic inflammation. Acute inflammation is a short-term process characterized by the classic signs of inflammation, i.e. swelling, redness, pain, heat, and loss of function, due to increase movement of plasma and leukocytes from blood into the injured tissues. Chronic inflammation is a pathological condition characterized by constructive inflammation and tissue destruction. Chronic inflammation is the main contributing factor of many degenerative diseases and cancers.

Anti-inflammatory medications could be classified into those of steroid and non-steroid. Steroid anti-inflammatory drugs such as glucocorticoids are the most effective medications in treatment of inflammatory diseases such as asthma, allergic rhinitis, Crohn's disease, colitis, ulcerative colitis, eczema, psoriasis, neurodermatitis and rheumatoid arthritis. Although the types of drugs have great effectiveness, they also possess unfavourable side effects. Nonsteroidal anti-inflammatory drugs (NSAIDS) are the most widely used anti-inflammatory medications. Aspirin (acetylesalicylic acid), one of the prototypical and the oldest of NSAID, is still extensively used in the world. NSAID can inhibit the synthesis of inflammatory mediators such as prostaglandins and thromboxanes via inhibition of cyclooxygenase-1 (COX-1) and COX-2. NASIDS are also demonstrated unfavourable effects when used greater concentrations or over long periods. It is well known that the use of these medicaments usually causes injuries of the gastric mucosa and bleeding of patients.

Drinking or local application of human or animal urine for medical purpose has been practiced for thousand years originated from China, India, Ancient Egypt, Greece, Rome and other countries. Urine was referred as “gold of the blood” and an “elixir of long life” indicating its therapeutic potential (see also Savica V, et al. Journal of Nephrology 2011; 24:123-5). Ancient Chinese medical texts recorded specific ways to use urine and even described how it can be purified into a powdered crystal (KWLL) to treat asthma which is still used in modern Chinese medicine (see also Lin C C, et al. Evidence-Based Complementary and Alternative Medicine 2013; in press, article ID 262391).Saharan Bedouins used urine to clean burns and wounds. It is the same medical practice described in the Ebers Papyrus of 1500 B C, one of the oldest surviving documents of Egyptian history (heartlandhealing.com).The Aztec civilization also used urine to heal wounds. Other cultures recommend drinking urine to increase fertility and stimulate sexuality (see also http://www.heartlandhealing.com/pages/archive/urine_therapy/). Up to date, urine therapy has been proven for treatments of many diseases such as multiple sclerosis, colitis, lupus, rheumatic arthritis, cancer, hepatitis, hyperactivity, pancreatic insufficiency, psoriasis, eczema, diabetes, herpes, mononucleosis, adrenal failure, allergy and asthma, etc (see also http://www.shirleys-wellness-café.com/UT/Urine.aspx).

Here are some examples using urine therapy recently reported
1. Martha Christy cured herself of a host of incapacitating illnesses, including intestinal inflammation or Crohn's disease, chronic fatigue syndrome, thyoid disorder, severe chronic kidney infections, food and chemical allergies and severe endometriosis (see also http://www.whale.to/a/urine1.html).
2. Beijing, Jun. 1, 2000. More than three million Chinese drink their own urine in the belief it is good for their health, the official Xinhua news agency reported. Participants were told that urine contains many active ingredients which strengthen the immune system (see also http://groups.yahoo.com/group/SalvationScience/message/1383?source=1&var=1) 3. Carrie Steele from Colorado, US, suffered a brain aneurysm after melanoma remission in 2006. After two years of struggling with painful headaches and feared her cancer would return, in 2008, she started to drink her own pee a cup every day. After 30 days, she claimed that the pain she had felt for two years was gone. Later, she drunk five cups of urine every day and even used urine to brush her teeth, used urine as a moisturiser, added it to her bath water. Despite not taking any medications since 2008, she said she has not suffered from any pain, and her cancer has not returned (see also http://www.thesun.co.uk/sol/homepage/features/4732396/Carrie-Steele-advocates-urine-therapy.html)

Several scientific articles also reported the benefits by using urine to treat various diseases. Wilson reported using urine therapy against allergic symptoms (see also Wilson CW. Medical hypotheses 1984; 13:99-107). Bercovitz reported using urine from pregnant mares to cure chronic duodenal ulcer (Bercovitz Z T. Gastroentrology 1954; 26:230-8). The scientific evidence by using purified urine crystal KWLL indicated that KWLL significantly reduced Dermatophagoides-pteronyssinus-induced airway hyperresponsiveness and inhibited eosinophil infiltration through the down-regulation of IL-5 expression in bronchoalvolar lavage fluid. KWLL also inhibited neutrophil recruitment by down-regulating IL-17A in bronchoalvolar lavage fluid. It also effectively diminished inflammatory cells, goblet cell hyperplasia, and mRNA expression of IL-6 and IL-17A in the lung (see also Lin C C, et al. Evidence-Based Complementary and Alternative Medicine 2013; in press, article ID 262391). However, up to date, there is no any single component or chemical from urine that has been claimed as an anti-inflammatory or anti-cancer medicament.

Although high dose of ammonium (NH₄⁺) or ammonia (NH₃) is known being toxic to skin, eye and respiratory duct (http://www.sciencelab.com; Martinelle K et al. 1993, J Biotechnol 30:339-50), some ammonium consisting compounds have been used widely in human for many years. For example, an injection of ammonium chloride has been used to maintain the acid-base balance in patients with hypochloremia states and metabolic alkalosis (see also Galla J H. J Am Soc Nephrol 2000; 11: 369-375). Ammonium chloride was also used as an expectorant for cough stop syrup (see also http://en.wikipedia.org/wiki/Ammonium chloride). An expectorant is a drug that stimulates, depresses or modifies the secretion from the bronchial or laryngeal mucus membranes and promotes its expulsion Ammonium tetrathiomolybdate was used to treat neurologic sign and syndromes caused by Wilson's disease (Brewer G J et al. 1994, Arch Neurol 51: 545-554) Ammonium hydroxide was previously used to adjusted pH of skin lotion containing 12% lactic acid (final concentration of ammonium is 1.8 M). This skin lotion was designed to heal excessively dry, chapped, itchy, or scaly skin. Prior art searching, there is no any ammonium compound been used for a medicament of anti-inflammation.

### SUMMARY OF INVENTION

Based on the efficacy of urine as an anti-inflammatory medicament and the composition of urine in previous studies, herein, intensive researches repeated by the present inventor have resulted in finding that the ammonium ion (10 mM of either ammonium chloride or ammonium acetate or ammonium sulfate, in which concentration is similar with normal urine from human being and animals) by topical or gargle or spray administration has excellent effects for dermatological inflammatory diseases such eczema, acnes, psoriasis, furuncles and other inflammatory diseases or syndrome such as pain, pharyngitis, periodontitis, arthritis, swell, redness and pain caused by tissue injury, and subcutaneous masses.

In a first aspect, the present invention provides compounds of general formula: (NH₄⁺)n-R Wherein “n” is optional number of 1 or more than 1. Wherein “R” presents any ion constituted ammonium salt with “n” of NH₄⁺ ion.

The ammonium salt [(NH₄⁺)n-R] may include a large of spectrum of compound consisting of one or more than one NH₄⁺ ions.

In another aspect, the invention is directed to a pharmaceutical compound comprising a ammonium salt [(NH₄⁺)n-R], as described generally herein, and an active ingredient as NH₄⁺. In a specific embodiment, the ammonium ion is useful in the treatment and prevention of human or animal inflammation related diseases.

According to still another aspect of the present invention, there is provided a method for treating and preventing inflammatory diseases in human being and animals, comprising the step of administering therapeutically effective dosage of the ammonium ion (NH₄⁺) to the patients in need of treatment.

### BRIEF DESCRIPTION OF THE FIGURES

The accompanying drawings are included to provide a further understanding of the invention, and are incorporated in and constitute a part of this specification.

FIG. 1 shows that treatment with 10 mM of NH₄Cl for 3 months cures psoriasis on both hands in patient A with psoriasis for 3 years.

FIG. 2 shows that treatment with 10 mM of NH₄Cl for 3 months cures psoriasis on both elbows in patient A with psoriasis for 3 years.

FIG. 3 shows that treatment with 10 mM of NH₄Cl for 2 months cures psoriasis on one hand in patient A with psoriasis for 3 years.

FIG. 4 shows that treatment with 10 mM of NH₄Cl for 3 months cure psoriasis on the back of the body in patient A with psoriasis for 3 years.

FIG. 5 shows that treatment with 10 mM of NH₄Cl for 4 months improve appearance of psoriasis on feet and both knees in patient A with psoriasis for 3 years.

### DETAILED DESCRIPTION OF THE INVENTION

I. Definitions

Unless defined otherwise, technical and scientific terms used herein have the same meaning as commonly understood by one of ordinary skill in the art.

As used herein, the term “anti-inflammation” refers to an ability to counteract, prevent, and/or reduce tissue inflammation caused by infection, disease, auto-antibody, and /or trauma. Anti-inflammatory compounds of the present invention possess such ability.

As used herein, the term “treatment”, “treating”, and like mean obtaining a desired pharmacologic and/or physiological effect. The desired effect maybe, prevention of a disease and/or may be therapeutic in terms of a partial or complete cure for a disease.

II. Methods and Formulations

The present invention is directed to a novel solution for treating inflammatory diseases. The formulation of the present invention comprising an ammonium salt (NH₄⁺) mixed with commercial available skin moisture lotion without specification for topical applications. The other formulation of the present invention comprising an ammonium salt (NH₄⁺) in H₂O for spray or rinse or gargle.

III. Dose

The phrase “pharmaceutically effective amount” is an art-recognized term, and refers to an amount of a compound that, when incorporated into a pharmaceutical formulation of the present invention, produces some desired effect to any medical treatment. The preferable concentration of ammonium to treat inflammatory diseases by topical, intranasl, sprayed and gargled delivery in the present invention is 10 mM (range from 1 mM to 100 mM).

The phrase “pharmaceutically acceptable” is art-recognized and refers to formulations and other materials and/or dosage forms which are suitable for use in contact with the tissues of human beings and animals without excessive toxicity, irritation, allergic response, or other problem or complications. The pharmaceutically acceptable concentration for ammonium salts used by either topical or gargle is 10 mM for final concentration in the formulation in the present invention. If the route of administration for ammonium salt has been changed such as oral or injection, the pharmaceutically acceptable concentration may be different.

In particular embodiments, a formulation is administered once a day. However, the formulations of the present invention may also be formulated for administration at any frequency of administration, including once a week, once every 3 days, once every 2 days, once a day, twice a day, three times a days and even greater frequency.

### SAMPLES

The following examples are provided to illustrate certain aspects of the present invention and to aid those of skilled in the art in practicing this invention. These examples are no way to be considered to limit the scope of the invention in any manner. However, it will be appreciated that those skilled in the art, on consideration of this disclosure, may make modifications and improvement within the spirit and scope of the present invention.

### Example 1

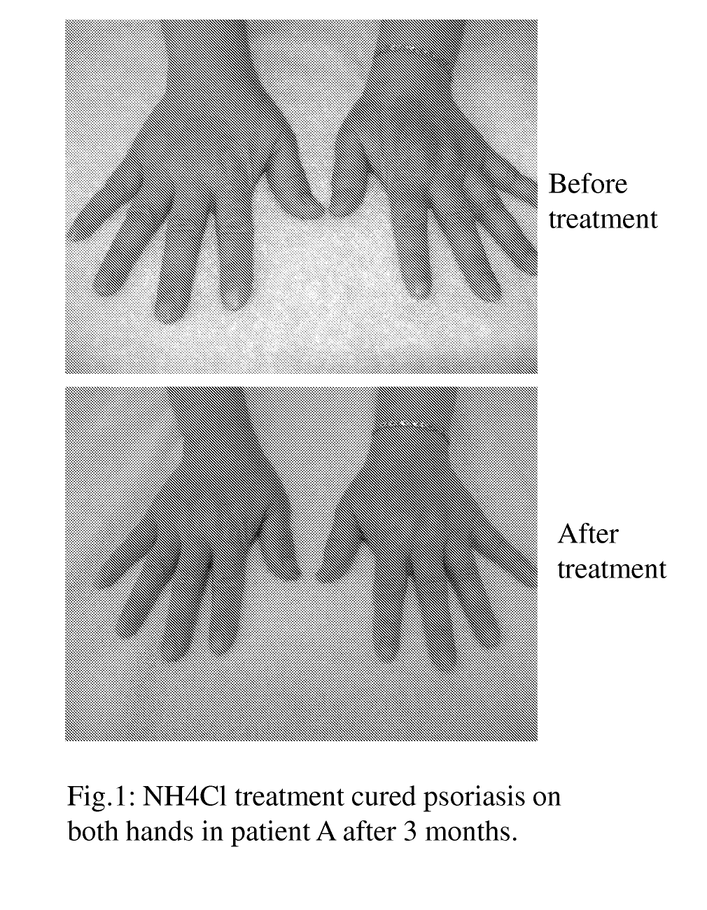

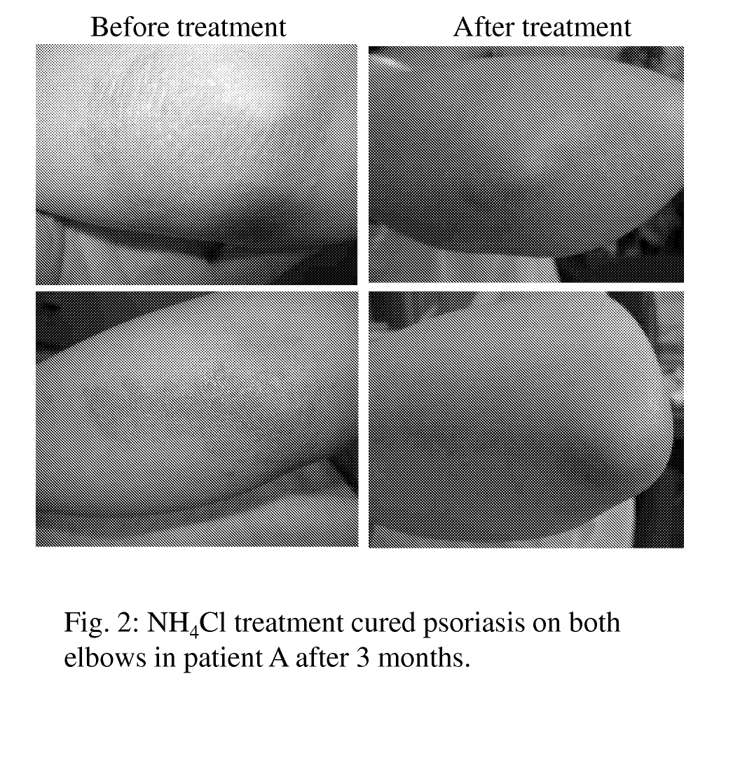

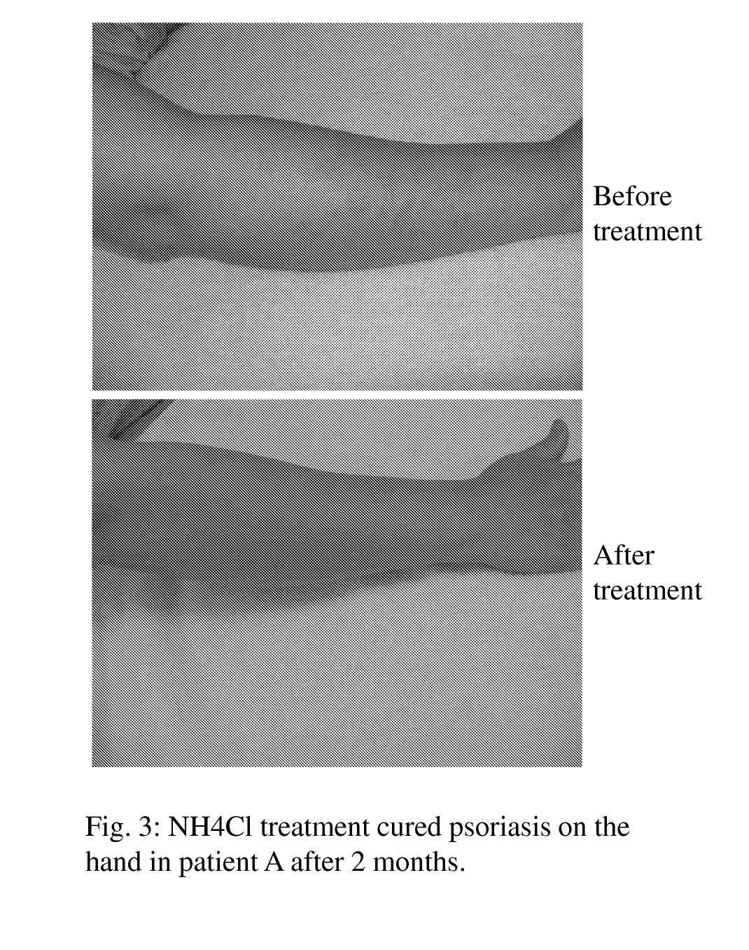

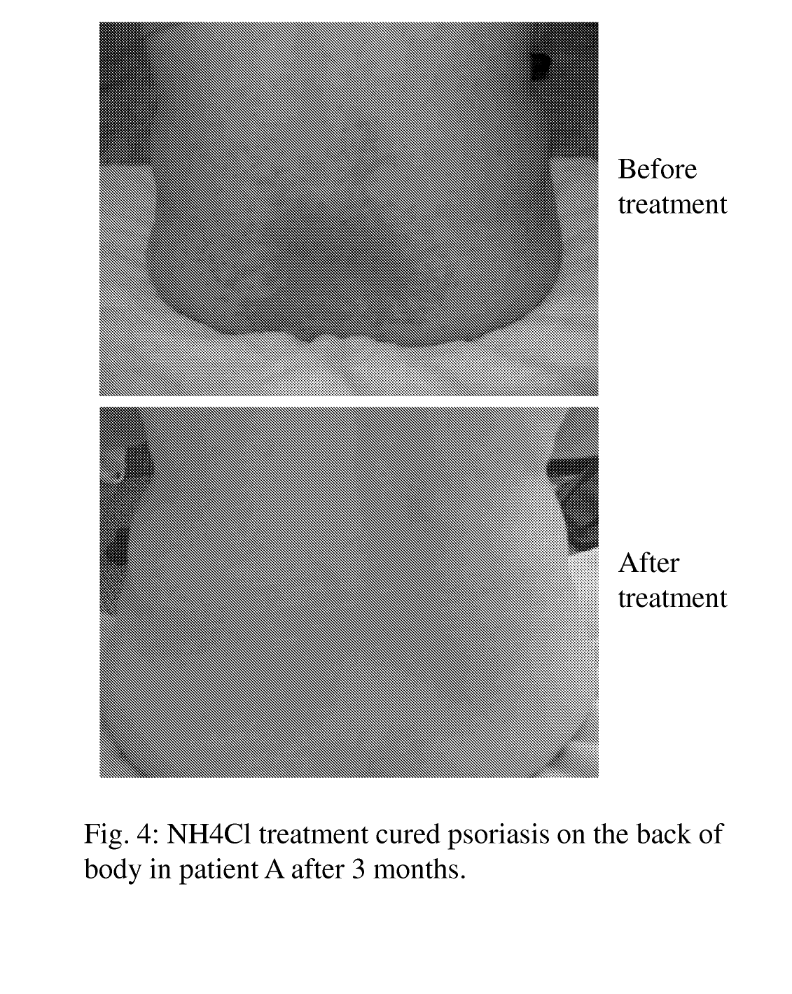

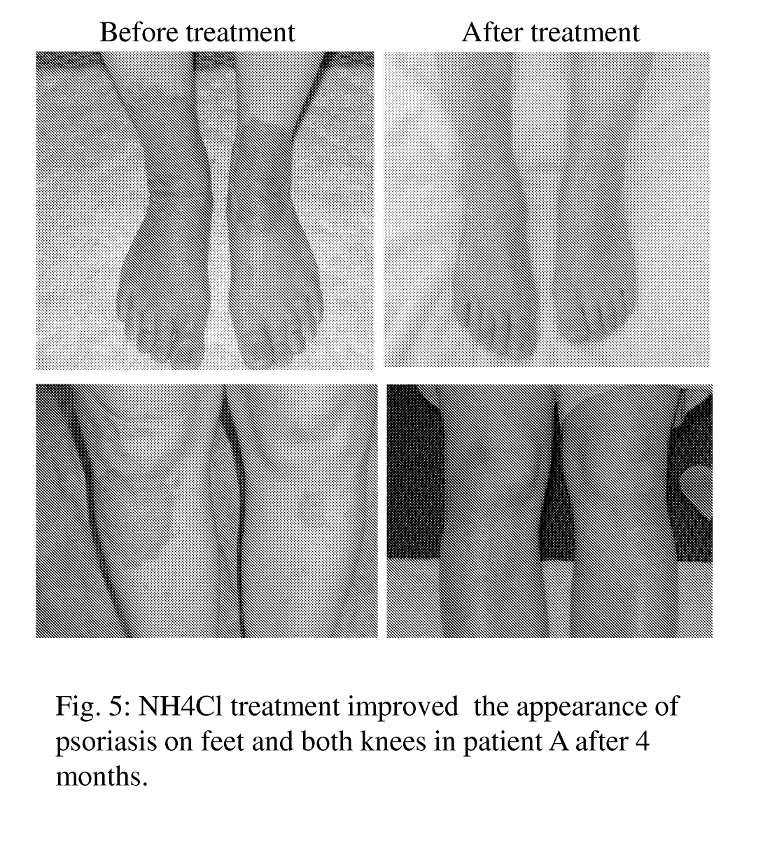

Patient A with psoriasis for 3 years was sprayed 10 mM of NH₄Cl solution once every 2 days toward the affected sites of disease (two hands, two elbows, one arm, back of body, two knees and two feet), without additional treatments. Photos were taken before and after treatments in indicated time points. FIGS. 1, 2, 3, 4 and 5 illustrated the results of the above-identified protocol used in this study.

### Example 2

NH₄Cl was mixed with commercial available skin moisture lotions at a final concentration of 10 mM and topically used to treat furuncles in a frequency of 3 times a day. After treatment for 2-3 days, furuncles were cured (pain was stopped, red was faded and nodule was faded).

### Example 3

NH₄Cl was mixed with skin moisture lotions at a final concentration of 10 mM and topically used to reduce pain of body in a frequency of once a day. After treatment for 2-3 days, pain would be completed stopped or significantly reduced.

### Example 4

NH₄Cl was mixed with skin moisture lotions at a final concentration of 10 mM and topically applied to treat acnes in a frequency of twice a day. After treatment for 3-5 days, acnes would be cured.

### Sample 5

NH₄Cl was mixed with skin moisture lotions at a final concentration of 10 mM and topically applied to treat subcutaneous masses in a frequency once a day. After treatment for 2-3 days, masses less than 1 cm would be disappeared.

### Sample 6

To treat acute or chronic pharyngitis or periodontitis, patients directly gargled 10 mM NH₄Cl in H₂O for 5 minutes twice a day. For acute pharyngitis or periodontitis, 2-3 days, disease wascured; for chronic pharyngiits or periodontitis, used for 1 week, pharyngitis or periodonitis would be cured.

### Example 7

NH₄Cl was mixed with skin moisture lotions to make a NH₄⁺ final concentration of 10 mM and used topically on face once a week could prevent acnes or furuncles.

### Example 8

NH₄Cl was mixed with skin moisture lotions to make a NH₄⁺ final concentration of 10 mM and used topically on the suffering sites in patients with either acute or chronic arthritis once a day. Swell, redness and pain would be cured or significantly reduce.

### Example 9

NH₄Cl was mixed with skin moisture lotions to make a NH₄⁺ final concentration of 10 mM and used topically on the suffering sites of acute or chronic injured tissue once a day. Swell, redness and pain would be cured or significantly reduce.

1. A method for treatment of inflammation and related swell, pain, edema, adhesions and combinations thereof comprising administration to a patient in need thereof a medicament consisting essentially of a therapeutically effective amount of ammonium ion (NH₄⁺) represented by Formula (NH₄⁺)ₙ—R. Wherein “n” is optional number of 1 or more than 1.

2. The method according to claim 1, wherein the medicament is administrated in preparation selected from the group consisting of an oral preparation, a preparation for injection, or a topical preparation.

3. The method according to claim 2, wherein the topical preparation is selected from the group consisting of an ointment, a liquid extract, a plaster, a wet towel, a liniment, and a paint.

4. A health food adjuvant or health beverage comprising a therapeutically effective amount of ammonium ion (NH₄⁺) as an active ingredient.

5. The health food adjuvant or health beverage according claim 4, wherein the health food adjuvant or health beverage is used for prevention or treatment of inflammation.

6. A food additive comprising a therapeutically effective amount of ammonium ion (NH₄⁺) as an active ingredient.

7. The food additive according claim 6, wherein the food additive is used for prevention or treatment of inflammation.

8. An animal feed composition comprising a therapeutically effective amount of ammonium ion (NH₄⁺) as an active ingredient.

9. The animal feed composition according to claim 8, wherein the animal feed composition is used for prevention or treatment of inflammation.

10. A functional cosmetic composition comprising a therapeutically effective amount of ammonium ion (NH₄⁺) as an active ingredient.

11. The functional cosmetic composition according to claim 10, wherein the functional cosmetic composition is used for prevention or treatment of inflammation.
